(19) United States
(12) Patent Application Publication
(10) Pub. No.: US 2010/0202978 A1
(43) Pub. Date: Aug. 12, 2010

(54) FOAMABLE BENZOYL PEROXIDE COMPOSITIONS FOR TOPICAL ADMINISTRATION

(76) Inventors: Ronald M. Gurge, Franklin, MA (US); Mark W. Trumbore, Westford, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/546,189

(22) Filed: Aug. 24, 2009

Related U.S. Application Data
(60) Provisional application No. 61/152,022, filed on Feb. 12, 2009.

(51) Int. Cl. A61K 9/12; A61P 17/00

(52) U.S. Cl. 424/45

(57) ABSTRACT

Described herein are benzoyl peroxide compositions useful in the treatment of acne and other skin conditions, which exhibit enhanced stability, even under accelerated conditions. The compositions also exhibit reduced color formation, reduced irritation, and enhanced moisturizing properties. They can be formulated into a topical aerosol foam with inert, non-flammable propellants, such as hydrofluoroalkanes, and may be used in cosmetics or pharmaceuticals. Additionally, methods of formulating these compositions are described.

### RELATED APPLICATIONS

This application claims the benefit of priority to U.S. Provisional Patent Application Ser. No. 61/152,022, filed Feb. 12, 2009.

### BACKGROUND

Acne is a pleomorphic skin disease characterized by blackheads, whiteheads, papules, pustules, and cysts. The lesions are often contaminated with bacteria, which can lead to secondary infections. The appearance of acne ranges from slight skin irritation to pitting. In extreme cases, acne leads to the development of disfiguring scars.

Therapeutic approaches to treating acne include prevention of obstruction of the follicular duct, reopening the duct if it has become blocked, combating any infecting bacteria, or reducing the thickened sebum, and combinations of these approaches. The horny outer layer of the skin (stratum corneum) is formed of dead cells composed largely of keratin. Therapeutic agents which act to prevent the obstruction of the follicular duct by the removal of excess keratin are known as keratolytic agents. Salicylic acid, sulfur, and resorcinol have been employed as keratolytic agents in the management of acne for at least 100 years.

Additionally, benzoyl peroxide (BPO), an organic peroxide, has been employed as a keratolytic drug in the topical treatment of skin lesions, such as acne, burns, varicose ulcers, sycosis vulgaris, and seborrhea for the past sixty years. BPO has proven to be an effective topical non-prescription medication for acne. Benzoyl peroxide [(C₆H₅CO)₂O₂] is a colorless, odorless, tasteless, crystalline solid, which is a powerful oxidizing agent, yet nontoxic to man.

While benzoyl peroxide is recognized by the F.D.A. as effective in concentrations between 2.5% and 10% for the local treatment of skin lesions, such as those developed in acne or seborrhea, it has the adverse side effect of causing contact irritation and dryness. Additionally, a small percentage of the population experiences a mild allergic reaction to certain benzoyl peroxide formulations. Furthermore, BPO products have a characteristic odor and can stain clothing. These negative aesthetic product attributes can have an effect on patient compliance. Accordingly, some patients are unable to derive the benefits provided by BPO acne therapy.

Additionally, BPO is extremely unstable. Under most conditions, BPO decomposes in both solid and solution state; the rate of degradation increases exponentially with an increase in temperature. Due to its strong oxidative capabilities, BPO reacts unpredictably with other compounds and it is very difficult to obtain stabilized formulations. Therefore, common shortcomings of products containing BPO (alone or in combination with additional active ingredients) are falloff of activity and efficacy with time, and their associated short shelf lives.

A 10% benzoyl peroxide foam is available. This product utilizes propane and butane as propellants, both of which are extremely flammable. The product, therefore, combines two incompatible hazardous classes of compounds in an aerosol container: an oxidizer (benzoyl peroxide); and flammable hydrocarbons (butane and propane). This creates a safety issue and a potential explosion hazard within the product container. The expiration dating on the product indicates that the product has a limited shelf life, possibly due to the poor stability of the formulation when stored under pressurized conditions.

Consequently, there is a need for cosmetically-elegant, stable, topical foam compositions containing BPO. It is therefore an object of the present invention to provide topical aerosol compositions containing BPO with improved chemical and physical stability.

### SUMMARY OF THE INVENTION

One aspect of the invention relates to compositions comprising benzoyl peroxide, which exhibit enhanced stability, even under accelerated conditions. The compositions also exhibit reduced color formation and enhanced moisturizing properties, as compared to known formulations of benzoyl peroxide. The compositions can be formulated into a topical aerosol foam and used in cosmetics or pharmaceuticals. The compositions are useful in the treatment of acne and other skin conditions. Additionally, methods of formulating these compositions are described.

### BRIEF DESCRIPTION OF THE FIGURES

FIG. 1 depicts formulation E (from Table 1; BPO (1 g)+water (9 g)+HFA 134a (12 g)) after storage under accelerated conditions (6 months @ 30° C.).

FIG. 2 depicts formulation G (from Table 1; BPO (1 g)+water (9 g)+HFA 134a (12 g)+argon purge, 2 min) after storage under accelerated conditions (6 months @ 30° C.).

FIG. 3 depicts the average a*-value over time for subjects (n=10) treated with a blank (diamond), an aerosol foam vehicle (square), a 3.3% BPO aerosol foam (triangle), and a 5.3% BPO aerosol foam (X). The subjects were treated twice daily.

FIG. 4 depicts baseline normalized stratum corneum moisture values following treatment with 5.3% BPO aerosol foam (diamond), 5% BPO/1% Clindamycin Gel (square), and generic 5% BPO gel (triangle); n=10. Here, “*” indicates a statistically significant difference (p<0.05) at the 95% confidence interval.

### DETAILED DESCRIPTION OF THE INVENTION

In one embodiment, the invention relates to aqueous compositions of BPO which exhibit enhanced chemical and physical stability and reduced color formation. In one embodiment, the compositions feature a semi-solid oil phase. In one embodiment, the compositions do not contain volatile lower alcohols. In one embodiment, the compositions comprise an aerosol propellant. In one embodiment, the aerosol propellant is a hydrofluoroalkane propellant (HFA). In one embodiment, the compositions produce a foam upon actuation of an aerosol container charged with the composition. In one embodiment, the foams are stable against collapse. In one embodiment, the foams rub-in quickly without a greasy residue. In one embodiment, the foam is moisturizing. In one embodiment, the foam is non-irritating.

### Propellants

There are several possible choices of propellants for a BPO aerosol foam, including, but not limited to, CFCs, hydrocarbons, compressed gases, and HFAs. The Montreal Protocol has banned the use of CFCs (chlorofluorocarbons) due to their ability to deplete the ozone layer. Montreal Protocol on Substances that Deplete the Ozone Layer, United Nations Environmental Programme, 1987. Alternatively, hydrocarbon propellants demonstrate very low reactivity and good resistance to free-radical attack. However, hydrocarbon propellants are highly flammable and it would be undesirable and hazardous to combine these propellants with benzoyl peroxide, a strong oxidizer, in an aerosol foam system. The chemical classes of “oxidizer” and “flammable” are known to be incompatible. Finally, compressed inert gases, such as nitrogen and carbon dioxide, can be used as an aerosol propellant. While offering good chemical stability due to their non-reactivity, they are unable to deliver consistent product delivery throughout the life of the aerosol can due to their high vapor pressures.

Another option is HFAs (hydrofluoroalkanes, also known as hydrofluorocarbons, or HFCs). These propellants are pharmaceutically acceptable, generally non-reactive, and ozone-friendly. However, in the presence of free-radicals, HFA propellants are susceptible to free-radical attack. Labile hydrogen atoms can be abstracted due to the electron-withdrawing effects of the fluorine atoms, resulting in a hydrofluorocarbon radical. HFAs have been shown to form a hydro-fluorocarbon radical in the presence of hydroxyl radical (.OH). Maricq, M. M. and Szente, J. J. Flash Photolysis—Time-Resolved UV Spectroscopy of the CF₃CFHO₂ Self-Reaction. J. Phys. Chem. 1992, 96, 10862-10868. Post-initiation, the resulting reactive, hydrofluorocarbon radical has the potential to promote a variety of cascading, free-radical decompositions of other compounds in its presence.

A series of experiments were completed which confirmed the chemical incompatibility of benzoyl peroxide with HFAs. A commercially available 10% benzoyl peroxide wash was stored with and without HFA 134a propellant at 40° C. for two months. The sample stored with the HFA propellant showed a 10% drop in benzoyl peroxide assay, while the sample stored without the propellant showed no decrease in assay. Storage of a 10% aqueous solution of BPO in the presence of HFA 134a propellant results in a 30.80% average loss of BPO when stored for three months at 40° C. These experiments (outlined in Example 1) confirm that BPO is chemically unstable in the presence of 1,1,1,2-tetrafluoroethane (HFA 134a) alone.

It was surprisingly discovered that the chemical structure of the fluorinated propellant has an effect on benzoyl peroxide stability. Benzoyl peroxide was found to be more stable in the presence of fluorinated propellants with a labile proton on a secondary)(2° carbon (HFA 227) than fluorinated propellants with a labile proton on a primary)(1° carbon (HFA 134a).

Due to the complexities involved in obtaining a stable benzoyl peroxide system, solving the complex stability issue of benzoyl peroxide in the presence of fluorinated aerosol propellants is not a routine task.

### Antioxidants and Chelating Agents

The decomposition of benzoyl peroxide is complex and susceptible to many factors, some of them still unknown. Gupta, V. D. Effect of Some Formulation Adjuncts on the Stability of Benzoyl Peroxide. J. Pharm. Sci. 1982, 71(5), 585-587. The benefits of adding stabilizer(s) are the subject of debate. It has been suggested that addition of chelating agents (citric acid or EDTA) might have an adverse effect on the stability of benzoyl peroxide. Bollinger, J. N.; Lewis, D.; and Mendez V. M. Benzoyl Peroxide Stability in Pharmaceutical Gel Preparations. J. Pharm. Sci. 1977, 66, 718. Yet some commercial products add these chelating agents in an effort to improve stability. In addition, the use of antioxidants to improve BPO stability has yielded unpredictable results. U.S. Pat. No. 7,153,888; incorporated by reference. The reasons that certain antioxidants stabilize BPO, while others do not, are still unclear. In addition, excipient compatibility with benzoyl peroxide has been shown to be unpredictable.

Solutions of BPO in the absence of aerosol propellants have been stabilized by use of antioxidants. It has been shown that a ratio of approximately 5:1 BPO/antioxidant was required for stabilization. Smaller proportions of antioxidants failed to stabilize BPO.

### Stabilization of Exemplary Compositions of the Invention

Surprisingly, it has been found that it is possible to develop a stable aerosol foam formulation containing benzoyl peroxide and fluorinated propellants. In one embodiment, the invention relates to the formation of a stable benzoyl peroxide aerosol foam formulation, thus overcoming the expected and well-known stability issues associated with the fluorinated propellant system.

In one embodiment, the compositions are formulated such that the chemical instability due to the nature of the propellant is reduced. For example, compositions were formulated with the addition of antioxidants to the concentrate. Additionally, the air in the container headspace was replaced with an inert gas (argon). Compositions formulated in this way exhibited improved BPO stability in the presence of HFA propellants (HFA 134a and HFA 227). As a result, addition of argon and antioxidants to the system results in only a 23.2% average loss of BPO when stored for three months at accelerated conditions (40° C./75% relative humidity). See Tables 1 and 2. In one embodiment, the ratio of BPO/antioxidant utilized was 100:1 (5% BPO formulations) or 200:1 (10% BPO formulations), a ratio 20- to 40-times lower than the ratio previously reported to be required.

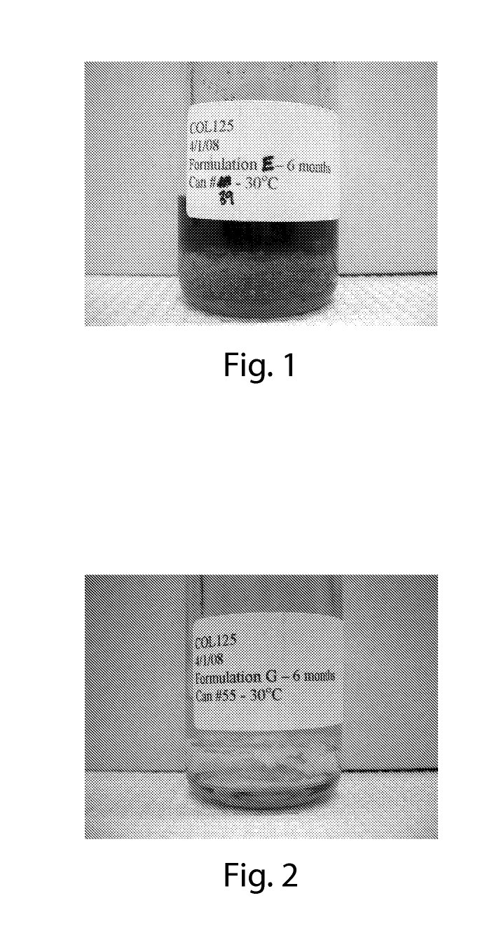

In addition, it was also surprisingly discovered that the color of the aerosol system containing argon/antioxidants was also significantly improved (colorless) when compared to the aerosol system without argon/antioxidants (dark brown). In one embodiment, the compositions are not colored, even after exposure to accelerated conditions. FIGS. 1 and 2 demonstrate the effect of inert gas on the color of the aqueous 10% BPO/HFA 134a system.

### Moisturization and Irritation

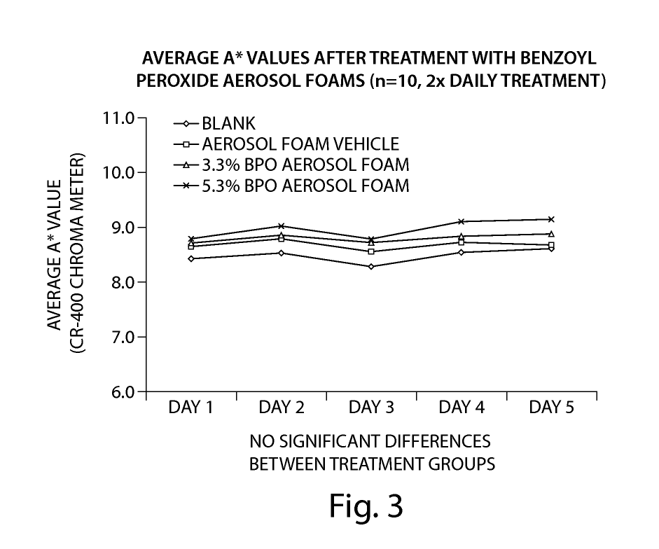

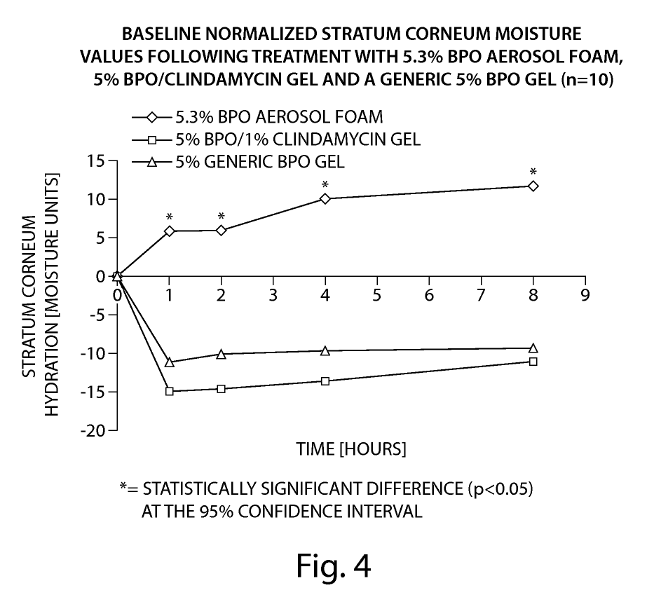

Topical formulations of benzoyl peroxide are known to be generally irritating and lack the ability to hydrate skin. These two negative attributes can lead to reduced patient compliance with its concomitant impact of therapeutic response. It was surprisingly found that embodiments of the inventive aerosol foam formulations of benzoyl peroxide were no more irritating than vehicle control and demonstrated similar levels of erythema as intact untreated skin. FIG. 3 shows the level of skin irritation as measured by redness value for untreated, vehicle-treated, and benzoyl peroxide foam-treated skin. It was also surprisingly discovered that, in contrast to marketed benzoyl peroxide products of similar potency, embodiments of the inventive aerosol foam formulations of benzoyl peroxide had the ability to moisturize skin. FIG. 4 shows the ability of an aerosol foam formulation of benzoyl peroxide to improve skin moisturization. This figure also shows the lack of moisturization activity in marketed products.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Exemplary Constituents of Compositions of the Present Invention

Exemplary identities of various constituents of the compositions of the present invention are described below.

### 1. Propellants

In one embodiment, the propellant is a HFA or a mixture of one or more hydrofluorocarbons. Suitable hydrofluorocarbons include 1,1,1,2-tetrafluoroethane (HFA 134a); 1,1,1,2,3,3,3-heptafluoropropane (HFA 227); and mixtures and admixtures of these and other HFAs that are currently approved or may become approved for medical use are suitable. Hydrocarbon as well as chlorofluorocarbon (CFC) propellants can also be used in the present invention.

### 2. Benzoyl Peroxide

Benzoyl peroxide works as a peeling agent, increasing skin turnover and clearing pores. Additionally, it is an antibacterial. This combination of uses makes it an ideal treatment for acne and other types of skin ulcers.

### 3. Other Active Agents

One or more additional active agents may be present in the composition. These include any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones.

3.1 Antibiotics

Representative antibiotics include, without limitation, octopirox, erythromycin, zinc, tetracyclin, triclosan, azelaic acid and its derivatives, phenoxy ethanol and phenoxy proponol, ethyl acetate, clindamycin and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin.

3.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents.

3.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

3.4 Anesthetics

Suitable anesthetics include the aminoacylanilide compounds such as lidocaine, prilocalne, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

3.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

3.6 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid and its derivatives (e.g., cis and trans, esters), alpha hydroxy acids, beta hydroxy acids, coal tar, and combinations thereof.

3.7 Other Agents

Suitable other agents include, but are not limited to, skin soothing agents, deodorant agents, antiperspirants, sun screening agents, sunless tanning agents, vitamins, hair conditioning agents, anti-irritants, anti-aging agents, and combinations thereof.

Examples of skin soothing agents include, but are not limited to, allantoin, aloe, avocado oil, green tea extract, hops extract, chamomile extract, colloidal oatmeal, calamine, cucumber extract, and combinations thereof.

Examples of vitamins include, but are not limited to, vitamins A, D, E, K, and combinations thereof.

Examples of sunscreens include, but are not limited to, p-aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide, 4-methylbenzylidene camphor, Methylene Bis-Benzotriazolyl Tetramethylbutylphenol, Bis-Ethylhexyloxyphenol Methoxyphenyl Triazine, Terephthalylidene Dicamphor Sulfonic Acid, Drometrizole Trisiloxane, Disodium Phenyl Dibenzimidazole Tetrasulfonate, Diethylamino Hydroxybenzoyl Hexyl Benzoate, Octyl Triazone, Diethylhexyl Butamido Triazone, Polysilicone-15, and combinations thereof

### 4. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as a-tocopherol), sodium ascorbate/ascorbic acid, ascorbyl palmitate, propyl gallate, and chelating agents like EDTA (e.g., disodium EDTA), citric acid, and sodium citrate.

In addition, combinations or mixtures of these preservatives or anti-oxidants may also be used in the formulations of the present invention.

### 5. Surfactants and Emulsifiers

Many topical formulations contain chemical emulsions which use surface active ingredients (emulsifiers) to disperse dissimilar chemicals in a particular solvent system. For example, most lipid-like (oily or fatty) or lipophilic ingredients do not uniformly disperse in aqueous solvents unless they are first combined with emulsifiers which form microscopic aqueous soluble micelles that contain a lipid-soluble interior and an aqueous-soluble exterior, resulting in an oil-in-water emulsion. In order to be soluble in aqueous media, a molecule must be polar or charged so as to favorably interact with water molecules which are also polar. Similarly, to dissolve an aqueous-soluble polar or charged ingredient in a largely lipid or oil-based solvent, an emulsifier is typically used which forms stable micelles that contain the aqueous-soluble components in the micelle interior while the exterior of the micelle is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion micelle. Emulsion destabilization results in the aqueous and lipophilic ingredients separating into two layers, potentially destroying the commercial value of a topical product.

Surfactants suitable for use in the present invention may be ionic or non-ionic. These include, but are not limited to: polysorbates (Polysorbate 20, Polysorbate 40, Polysorbate 60, Polysorbate 80), steareth-10, sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, and methylbenzethonium chloride. Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate and PEG-100 stearate, steareth-2 and steareth-20, or combinations/mixtures thereof, as well as cationic emulsifiers like stearamidopropyl dimethylamine and behentrimonium methosulfate, or combinations/mixtures thereof

### 6. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 7. Moisturizers and Emollients

One of the most important aspects of topical products in general, and cosmetic products in particular, is the consumer's perception of the aesthetic qualities of a product. For example, while petrolatum is an excellent moisturizer and skin product, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients for use in the formulations of the present invention include, but are not limited to, glycerol, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, and dicaprylate/dicaprate.

In addition, appropriate combinations and mixtures of any of these moisturizing agents and emollients may be used in accordance with the present invention.

### 8. Viscosity Modifiers

Suitable viscosity adjusting agents (i.e., thickening and thinning agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these vicosity adjusters may be utilized according to the present invention

### 9. Additional Constituents

Additional constituents suitable for incorporation into the emulsions of the present invention include, but are not limited to: skin protectants, adsorbents, demulcents, emollients, moisturizers, buffering agents, sustained release materials, solubilizing agents, skin-penetration agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, and pH adjusters (e.g., citric acid, sodium hydroxide, and sodium phosphate).

Suitable fragrances and colors may be used in the formulations of the present invention. Examples of fragrances and colors suitable for use in topical products are known in the art.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### 10. Purging Gases

In one embodiment, the air in the container charged with the composition is replaced by an inert gas. In certain embodiments, the inert gas is selected from the group consisting of argon, nitrogen, and mixtures thereof.

### Exemplary Compositions

In one embodiment, the invention relates to a composition comprising a concentrate and a propellant, wherein

the concentrate comprises
an amount of benzoyl peroxide, wherein the amount of benzoyl peroxide is about 0.5% to about 15% by weight of the concentrate;
an amount of water, wherein the amount of water is about 55% to about 95% by weight of the concentrate; and
an amount of an antioxidant or a preservative, wherein the amount of the antioxidant or the preservative is about 0.01% to about 5% by weight of the concentrate; and

the propellant is a hydrofluoroalkane propellant.

In one embodiment, the invention relates to a composition consisting essentially of a concentrate and a propellant, wherein

the concentrate comprises
an amount of benzoyl peroxide, wherein the amount of benzoyl peroxide is about 0.5% to about 15% by weight of the concentrate;
an amount of water, wherein the amount of water is about 55% to about 95% by weight of the concentrate; and
an amount of an antioxidant or a preservative, wherein the amount of the antioxidant or the preservative is about 0.05% to about 5% by weight of the concentrate; and

the propellant is a hydrofluoroalkane propellant.

In one embodiment, the invention relates to a composition consisting of a concentrate and a propellant, wherein

the concentrate comprises
an amount of benzoyl peroxide, wherein the amount of benzoyl peroxide is about 0.5% to about 15% by weight of the concentrate;
an amount of water, wherein the amount of water is about 55% to about 95% by weight of the concentrate; and
an amount of an antioxidant or a preservative, wherein the amount of the antioxidant or the preservative is about 0.1% to about 5% by weight of the concentrate; and

the propellant is a hydrofluoroalkane propellant.

In one embodiment, the invention relates to the above-mentioned composition, wherein the hydrofluoroalkane propellant is 1,1,1,2-tetrafluoroethane, 1,1,1,2,3,3,3-heptafluoropropane, or a mixture thereof.

In one embodiment, the invention relates to any one of the above-mentioned compositions, wherein the hydrofluoroalkane propellant is 1,1,1,2-tetrafluoroethane.

In one embodiment, the invention relates to any one of the above-mentioned compositions, wherein benzoyl peroxide is present in an amount from about 1.0% to about 13% by weight of the concentrate. In one embodiment, the invention relates to any one of the above-mentioned compositions, wherein benzoyl peroxide is present in an amount from about 2.5% to about 12% by weight of the concentrate. In one embodiment, the invention relates to any one of the above-mentioned compositions, wherein benzoyl peroxide is present in an amount from about 4.0% to about 7.0% by weight of the concentrate. In one embodiment, the invention relates to any one of the above-mentioned compositions, wherein benzoyl peroxide is present in about 2.5%, about 3.0%, about 3.5%, about 4.0%, about 5.0%, about 6.0%, about 7.0%, about 8.0%, about 9.0%, about 10.0%, about 11%, or about 12% by weight of the concentrate. In one embodiment, the invention relates to any one of the above-mentioned compositions, wherein benzoyl peroxide is present in about 5.3% by weight of the concentrate.

In one embodiment, the invention relates to any one of the above-mentioned compositions, wherein water is present in an amount from about 60% to about 90% by weight of the concentrate. In one embodiment, the invention relates to any one of the above-mentioned compositions, wherein water is present in an amount from about 65% to about 90% by weight of the concentrate. In one embodiment, the invention relates to any one of the above-mentioned compositions, wherein water is present in an amount from about 72% to about 82% by weight of the concentrate. In one embodiment, the invention relates to any one of the above-mentioned compositions, wherein water is present in about 65%, about 70%, about 75%, about 80%, about 85%, or about 90% by weight of the concentrate. In one embodiment, the invention relates to any one of the above-mentioned compositions, wherein water is present in about 77% by weight of the concentrate.

In one embodiment, the invention relates to any one of the above-mentioned compositions, wherein the antioxidant or the preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, sodium ascorbate, ascorbyl palmitate, propyl gallate, disodium EDTA, and mixtures thereof.

In one embodiment, the invention relates to any one of the above-mentioned compositions, wherein the antioxidant or the preservative is methylparaben, propylparaben, butylated hydroxytoluene, disodium EDTA, citric acid, sodium citrate, or a mixture thereof.

In one embodiment, the invention relates to any one of the above-mentioned compositions, wherein the antioxidant or the preservative is present in an amount from about 0.05% to about 3.0% by weight of the concentrate. In one embodiment, the invention relates to any one of the above-mentioned compositions, wherein the antioxidant or the preservative is present in an amount from about 0.1% to about 2.0% by weight of the concentrate. In one embodiment, the invention relates to any one of the above-mentioned compositions, wherein the antioxidant or the preservative is present in an amount from about 0.5% to about 1.9% by weight of the concentrate. In one embodiment, the invention relates to any one of the above-mentioned compositions, wherein the antioxidant or the preservative is present in about 0.1%, about 0.2%, about 0.3%, about 0.4%, about 0.5%, about 0.6%, about 0.7%, about 0.8%, about 0.9%, about 1.0%, about 1.5%, or about 2.0% by weight of the concentrate. In one embodiment, the invention relates to any one of the above-mentioned compositions, wherein the antioxidant or the preservative is present in about 1.2% by weight of the concentrate.

In one embodiment, the invention relates to any one of the above-mentioned compositions, wherein the concentrate further comprises an emollient or a moisturizer.

In one embodiment, the invention relates to the above-mentioned composition, wherein the emollient or the moisturizer is selected from the group consisting of glycerol, PPG-15 stearyl ether, lanolin alcohol, lanolin, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, lactic acid, butylene glycol, sodium PCA, carbowax 200, carbowax 400, carbowax 800, and mixtures thereof.

In one embodiment, the invention relates to the above-mentioned composition, wherein the emollient or the moisturizer is glycerol, dimethicone, C₁₂-C₁₅ alkyl benzoates, propylene glycol, or a mixture thereof.

In one embodiment, the invention relates to any one of the above-mentioned compositions, wherein the emollient or the moisturizer is present in an amount from about 5% to about 20% by weight of the concentrate. In one embodiment, the invention relates to any one of the above-mentioned compositions, wherein the emollient or the moisturizer is present in an amount from about 8% to about 17% by weight of the concentrate. In one embodiment, the invention relates to any one of the above-mentioned compositions, wherein the emollient or the moisturizer is present in an amount from about 9% to about 15% by weight of the concentrate. In one embodiment, the invention relates to any one of the above-mentioned compositions, wherein the emollient or the moisturizer is present in an amount from about 9% to about 13% by weight of the concentrate. In one embodiment, the invention relates to any one of the above-mentioned compositions, wherein the emollient or the moisturizer is present in about 9%, about 10%, about 11%, about 12%, about 13%, about 14%, or about 15% by weight of the concentrate. In one embodiment, the invention relates to any one of the above-mentioned compositions, wherein the emollient or the moisturizer is present in about 11% by weight of the concentrate.

In one embodiment, the invention relates to any one of the above-mentioned compositions, wherein the concentrate further comprises an emulsifier or a surfactant.

In one embodiment, the invention relates to the above-mentioned composition, wherein the emulsifier or the surfactant is selected from the group consisting of polysorbate 20, polysorbate 40, polysorbate 60, polysorbate 80, steareth-10, sodium dodecyl sulfate, lauryl dimethyl amine oxide, cetyltrimethylammonium bromide (CTAB), polyoxyethylene sorbitan, octoxynol, N,N-dimethyldodecylamine-N-oxide, hexadecyltrimethylammonium bromide (HTAB), polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, behentrimonium methosulfate, and mixtures thereof.

In one embodiment, the invention relates to the above-mentioned composition, wherein the emulsifier or the surfactant is cetostearyl alcohol, emulsifying wax, steareth-10, or a mixture thereof.

In one embodiment, the invention relates to any one of the above-mentioned compositions, wherein the emulsifier or the surfactant is present in an amount from about 2% to about 10% by weight of the concentrate. In one embodiment, the invention relates to any one of the above-mentioned compositions, wherein the emulsifier or the surfactant is present in an amount from about 3% to about 9% by weight of the concentrate. In one embodiment, the invention relates to any one of the above-mentioned compositions, wherein the emulsifier or the surfactant is present in an amount from about 4% to about 8% by weight of the concentrate. In one embodiment, the invention relates to any one of the above-mentioned compositions, wherein the emulsifier or the surfactant is present in an amount from about 3% to about 7% by weight of the concentrate. In one embodiment, the invention relates to any one of the above-mentioned compositions, wherein the emulsifier or the surfactant is present in about 4%, about 5%, about 6%, about 7%, or about 8% by weight of the concentrate. In one embodiment, the invention relates to any one of the above-mentioned compositions, wherein the emulsifier or the surfactant is present in about 5% by weight of the concentrate.

In one embodiment, the invention relates to a composition consisting essentially of a concentrate and a propellant, wherein

the concentrate consists essentially of
from about 0.5% to about 15% benzoyl peroxide by weight of the concentrate;
from about 55% to about 95% water by weight of the concentrate; and
from about 0.01% to about 5% of a first mixture by weight of the concentrate, wherein the first mixture comprises butylated hydroxytoluene, methylparaben, propylparaben, citric acid, sodium citrate, or disodium EDTA;
from about 5% to about 20% of a second mixture by weight of the concentrate, wherein the second mixture comprises dimethicone, C₁₂-C₁₅ alkyl benzoates, glycerol, or propylene glycol; and
from about 2% to about 10% of a third mixture by weight of the concentrate, wherein the third mixture comprises cetostearyl alcohol, emulsifying wax, or steareth-10; and

the propellant is a hydrofluoroalkane propellant, wherein the hydrofluoroalkane propellant is 1,1,1,2-tetrafluoroethane or 1,1,1,2,3,3,3-heptafluoropropane.

In one embodiment, the invention relates to any one of the above-mentioned compositions, wherein benzoyl peroxide is present in an amount from about 1.0% to about 13% by weight of the concentrate. In one embodiment, the invention relates to any one of the above-mentioned compositions, wherein benzoyl peroxide is present in an amount from about 2.5% to about 12% by weight of the concentrate. In one embodiment, the invention relates to any one of the above-mentioned compositions, wherein benzoyl peroxide is present in an amount from about 4.0% to about 7.0% by weight of the concentrate. In one embodiment, the invention relates to any one of the above-mentioned compositions, wherein benzoyl peroxide is present in about 2.5%, about 3.0%, about 3.5%, about 4.0%, about 5.0%, about 6.0%, about 7.0%, about 8.0%, about 9.0%, about 10.0%, about 11%, or about 12% by weight of the concentrate. In one embodiment, the invention relates to any one of the above-mentioned compositions, wherein benzoyl peroxide is present in about 5.3% by weight of the concentrate.

In one embodiment, the invention relates to any one of the above-mentioned compositions, wherein water is present in an amount from about 60% to about 90% by weight of the concentrate. In one embodiment, the invention relates to any one of the above-mentioned compositions, wherein water is present in an amount from about 65% to about 90% by weight of the concentrate. In one embodiment, the invention relates to any one of the above-mentioned compositions, wherein water is present in an amount from about 72% to about 82% by weight of the concentrate. In one embodiment, the invention relates to any one of the above-mentioned compositions, wherein water is present in about 65%, about 70%, about 75%, about 80%, about 85%, or about 90% by weight of the concentrate. In one embodiment, the invention relates to any one of the above-mentioned compositions, wherein water is present in about 77% by weight of the concentrate.

In one embodiment, the invention relates to any one of the above-mentioned compositions, wherein the first mixture is present in an amount from about 0.05% to about 3.0% by weight of the concentrate. In one embodiment, the invention relates to any one of the above-mentioned compositions, wherein the first mixture is present in an amount from about 0.1% to about 2.0% by weight of the concentrate. In one embodiment, the invention relates to any one of the above-mentioned compositions, wherein the first mixture is present in an amount from about 0.5% to about 1.9% by weight of the concentrate. In one embodiment, the invention relates to any one of the above-mentioned compositions, wherein the first mixture is present in about 0.1%, about 0.2%, about 0.3%, about 0.4%, about 0.5%, about 0.6%, about 0.7%, about 0.8%, about 0.9%, about 1.0%, about 1.5%, or about 2.0% by weight of the concentrate. In one embodiment, the invention relates to any one of the above-mentioned compositions, wherein the first mixture is present in about 1.2% by weight of the concentrate.

In one embodiment, the invention relates to any one of the above-mentioned compositions, wherein the second mixture is present in an amount from about 8% to about 17% by weight of the concentrate. In one embodiment, the invention relates to any one of the above-mentioned compositions, wherein the second mixture is present in an amount from about 9% to about 15% by weight of the concentrate. In one embodiment, the invention relates to any one of the above-mentioned compositions, wherein the second mixture is present in an amount from about 9% to about 13% by weight of the concentrate. In one embodiment, the invention relates to any one of the above-mentioned compositions, wherein the second mixture is present in about 9%, about 10%, about 11%, about 12%, about 13%, about 14%, or about 15% by weight of the concentrate. In one embodiment, the invention relates to any one of the above-mentioned compositions, wherein the second mixture is present in about 11% by weight of the concentrate.

In one embodiment, the invention relates to any one of the above-mentioned compositions, wherein the third mixture is present in an amount from about 3% to about 9% by weight of the concentrate. In one embodiment, the invention relates to any one of the above-mentioned compositions, wherein the third mixture is present in an amount from about 4% to about 8% by weight of the concentrate. In one embodiment, the invention relates to any one of the above-mentioned compositions, wherein the third mixture is present in an amount from about 3% to about 7% by weight of the concentrate. In one embodiment, the invention relates to any one of the above-mentioned compositions, wherein the third mixture is present in about 4%, about 5%, about 6%, about 7%, or about 8% by weight of the concentrate. In one embodiment, the invention relates to any one of the above-mentioned compositions, wherein the third mixture is present in about 5% by weight of the concentrate.

In one embodiment, the invention relates to a composition consisting essentially of a concentrate and a propellant, wherein

the concentrate consists essentially of
about 5.3% benzoyl peroxide by weight of the concentrate;
about 77% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.6% sodium citrate by weight of the concentrate;
about 0.05% citric acid by weight of the concentrate;
about 0.9% dimethicone by weight of the concentrate;
about 0.4% C₁₂-C₁₅ alkyl benzoates by weight of the concentrate;
about 7.5% glycerol by weight of the concentrate;
about 2.5% propylene glycol by weight of the concentrate;
about 2.2% cetostearyl alcohol by weight of the concentrate;
about 2.2% emulsifying wax by weight of the concentrate; and
about 0.9% steareth-10 by weight of the concentrate; and

the propellant is 1,1,1,2-tetrafluoroethane or 1,1,1,2,3,3,3-heptafluoropropane.

In one embodiment, the invention relates to a composition consisting essentially of a concentrate and a propellant, wherein

the concentrate consists essentially of
about 5.3% benzoyl peroxide by weight of the concentrate;
about 76.73% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.62% sodium citrate by weight of the concentrate;
about 0.05% citric acid by weight of the concentrate;
about 0.9% dimethicone by weight of the concentrate;
about 0.45% C₁₂-C₁₅ alkyl benzoates by weight of the concentrate;
about 7.5% glycerol by weight of the concentrate;
about 2.5% propylene glycol by weight of the concentrate;
about 2.25% cetostearyl alcohol by weight of the concentrate;
about 2.25% emulsifying wax by weight of the concentrate; and
about 0.9% steareth-10 by weight of the concentrate; and

the propellant is 1,1,1,2-tetrafluoroethane or 1,1,1,2,3,3,3-heptafluoropropane.

In one embodiment, the invention relates to a composition consisting essentially of a concentrate and a propellant, wherein

the concentrate consists essentially of
about 5.3% benzoyl peroxide by weight of the concentrate;
about 76.73% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.62% sodium citrate by weight of the concentrate;
about 0.05% citric acid by weight of the concentrate;
about 0.9% dimethicone by weight of the concentrate;
about 0.45% C₁₂-C₁₅ alkyl benzoates by weight of the concentrate;
about 7.5% glycerol by weight of the concentrate;
about 2.5% propylene glycol by weight of the concentrate;
about 2.25% cetostearyl alcohol by weight of the concentrate;
about 2.25% emulsifying wax by weight of the concentrate; and
about 0.9% steareth-10 by weight of the concentrate; and

the propellant is 1,1,1,2-tetrafluoroethane.

In one embodiment, the invention relates to a composition consisting essentially of a concentrate and a propellant, wherein

the concentrate consists essentially of
about 5% benzoyl peroxide by weight of the concentrate;
about 77% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 1.0% dimethicone by weight of the concentrate;
about 0.5% C₁₂-C₁₅ alkyl benzoates by weight of the concentrate;
about 7.5% glycerol by weight of the concentrate;
about 2.5% propylene glycol by weight of the concentrate;
about 2.5% cetostearyl alcohol by weight of the concentrate;
about 2.5% emulsifying wax by weight of the concentrate; and
about 1.0% steareth-10 by weight of the concentrate; and

the propellant is 1,1,1,2-tetrafluoroethane or 1,1,1,2,3,3,3-heptafluoropropane.

In one embodiment, the invention relates to a composition consisting essentially of a concentrate and a propellant, wherein

the concentrate consists essentially of
about 10% benzoyl peroxide by weight of the concentrate;
about 72% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 1.0% dimethicone by weight of the concentrate;
about 0.5% C₁₂-C₁₅ alkyl benzoates by weight of the concentrate;
about 7.5% glycerol by weight of the concentrate;
about 2.5% propylene glycol by weight of the concentrate;
about 2.5% cetostearyl alcohol by weight of the concentrate;
about 2.5% emulsifying wax by weight of the concentrate; and
about 1.0% steareth-10 by weight of the concentrate; and

the propellant is 1,1,1,2-tetrafluoroethane or 1,1,1,2,3,3,3-heptafluoropropane.

In one embodiment, the invention relates to a composition consisting of a concentrate and a propellant, wherein

the concentrate consists of
about 5.3% benzoyl peroxide by weight of the concentrate;
about 77% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.6% sodium citrate by weight of the concentrate;
about 0.05% citric acid by weight of the concentrate;
about 0.9% dimethicone by weight of the concentrate;
about 0.4% C₁₂-C₁₅ alkyl benzoates by weight of the concentrate;
about 7.5% glycerol by weight of the concentrate;
about 2.5% propylene glycol by weight of the concentrate;
about 2.2% cetostearyl alcohol by weight of the concentrate;
about 2.2% emulsifying wax by weight of the concentrate; and
about 0.9% steareth-10 by weight of the concentrate; and

the propellant is 1,1,1,2-tetrafluoroethane or 1,1,1,2,3,3,3-heptafluoropropane.

In one embodiment, the invention relates to a composition consisting of a concentrate and a propellant, wherein

the concentrate consists of
about 5.3% benzoyl peroxide by weight of the concentrate;
about 76.73% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.62% sodium citrate by weight of the concentrate;
about 0.05% citric acid by weight of the concentrate;
about 0.9% dimethicone by weight of the concentrate;
about 0.45% C₁₂-C₁₅ alkyl benzoates by weight of the concentrate;
about 7.5% glycerol by weight of the concentrate;
about 2.5% propylene glycol by weight of the concentrate;
about 2.25% cetostearyl alcohol by weight of the concentrate;
about 2.25% emulsifying wax by weight of the concentrate; and
about 0.9% steareth-10 by weight of the concentrate; and

the propellant is 1,1,1,2-tetrafluoroethane or 1,1,1,2,3,3,3-heptafluoropropane.

In one embodiment, the invention relates to a composition consisting of a concentrate and a propellant, wherein

the concentrate consists of
about 5.3% benzoyl peroxide by weight of the concentrate;
about 76.73% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.62% sodium citrate by weight of the concentrate;
about 0.05% citric acid by weight of the concentrate;
about 0.9% dimethicone by weight of the concentrate;
about 0.45% C₁₂-C₁₅ alkyl benzoates by weight of the concentrate;
about 7.5% glycerol by weight of the concentrate;
about 2.5% propylene glycol by weight of the concentrate;
about 2.25% cetostearyl alcohol by weight of the concentrate;
about 2.25% emulsifying wax by weight of the concentrate; and
about 0.9% steareth-10 by weight of the concentrate; and

the propellant is 1,1,1,2-tetrafluoroethane.

In one embodiment, the invention relates to a composition consisting of a concentrate and a propellant, wherein

the concentrate consists of
about 5% benzoyl peroxide by weight of the concentrate;
about 77% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 1.0% dimethicone by weight of the concentrate;
about 0.5% C₁₂-C₁₅ alkyl benzoates by weight of the concentrate;
about 7.5% glycerol by weight of the concentrate;
about 2.5% propylene glycol by weight of the concentrate;
about 2.5% cetostearyl alcohol by weight of the concentrate;
about 2.5% emulsifying wax by weight of the concentrate; and
about 1.0% steareth-10 by weight of the concentrate; and

the propellant is 1,1,1,2-tetrafluoroethane or 1,1,1,2,3,3,3-heptafluoropropane.

In one embodiment, the invention relates to a composition consisting of a concentrate and a propellant, wherein

the concentrate consists of
about 10% benzoyl peroxide by weight of the concentrate;
about 72% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 1.0% dimethicone by weight of the concentrate;
about 0.5% C₁₂-C₁₅ alkyl benzoates by weight of the concentrate;
about 7.5% glycerol by weight of the concentrate;
about 2.5% propylene glycol by weight of the concentrate;
about 2.5% cetostearyl alcohol by weight of the concentrate;
about 2.5% emulsifying wax by weight of the concentrate; and
about 1.0% steareth-10 by weight of the concentrate; and

the propellant is 1,1,1,2-tetrafluoroethane or 1,1,1,2,3,3,3-heptafluoropropane.

In one embodiment, the invention relates to any one of the above-mentioned compositions, wherein the composition is colorless.

In one embodiment, the invention relates to any one of the above-mentioned compositions, wherein the composition is in an aerosol container.

In one embodiment, the invention relates to any one of the above-mentioned compositions, wherein the composition is in an aerosol container, thereby forming a headspace of the aerosol container; and the headspace of the aerosol container is substantially free of oxygen.

In one embodiment, the invention relates to any one of the above-mentioned compositions, thereby forming a headspace of the aerosol container; and the headspace of the aerosol container consists essentially of argon.

In one embodiment, the invention relates to any one of the above-mentioned compositions, wherein when the aerosol container is actuated, the composition is expelled as a foam.

In one embodiment, the invention relates to any one of the above-mentioned compositions, wherein the composition is in an aerosol container. In one embodiment, the invention relates to any one of the above-mentioned compositions, wherein the composition is about 4% to about 50% propellant, by weight of the composition. In one embodiment, the invention relates to any one of the above-mentioned compositions, wherein the composition is about 5% to about 40% propellant, by weight of the composition. In one embodiment, the invention relates to any one of the above-mentioned compositions, wherein the composition is about 6% to about 30% propellant, by weight of the composition. In one embodiment, the invention relates to any one of the above-mentioned compositions, wherein the composition is about 6% to about 18% propellant, by weight of the composition. In one embodiment, the invention relates to any one of the above-mentioned compositions, wherein the composition is about 6%, about 7%, about 8%, about 9%, about 10%, about 11%, about 12%, about 13%, about 14%, about 15%, about 20%, about 25%, or about 30% propellant, by weight of the composition. In one embodiment, the invention relates to any one of the above-mentioned compositions, wherein the composition is about 12% propellant, by weight of the composition. In one embodiment, the invention relates to any one of the above-mentioned compositions, wherein about 6%, about 7%, about 8%, about 9%, about 10%, about 11%, about 12%, about 13%, about 14%, about 15%, about 20%, about 25%, or about 30% propellant, by weight of the composition, is required to deliver the concentrate as a stable foam.

In one embodiment, the invention relates to any one of the above-mentioned compositions, wherein the composition is in the form of a foam.

In one embodiment, the invention relates to any one of the above-mentioned compositions, wherein the composition produces a foam.

In one embodiment, the invention relates to any one of the above-mentioned compositions, wherein the foam is produced by actuation of an aerosol container comprising the composition.

In one embodiment, the invention relates to any one of the above-mentioned compositions, wherein the foam is non-irritating when applied to the skin of a subject.

In one embodiment, the invention relates to any one of the above-mentioned compositions, wherein the foam is moisturizing over a period of at least 8 hours when applied to the skin of a subject.

In one embodiment, the invention relates to any one of the above-mentioned compositions, wherein the composition does not comprise methanol, ethanol, propanols, or butanols.

In one embodiment, the invention relates to any one of the above-mentioned compositions, wherein the composition does not comprise methane, ethane, propane, butane, pentane, or hexane.

In one embodiment, the invention relates to any one of the above-mentioned compositions, wherein the composition is non-irritating when applied to the skin.

In one embodiment, the invention relates to any one of the above-mentioned compositions, wherein the composition is moisturizing when applied to the skin. In one embodiment, when applied to the skin, the composition is moisturizing over a period of at least 4, at least 6, at least 8, at least 10, or at least 12 hours. In one embodiment, when applied to the skin, the composition is moisturizing over a period of up to about 24 hours. In one embodiment, when applied to the skin, the composition is moisturizing over a period of up to about 48 hours. In one embodiment, when applied to the skin, the composition is moisturizing over a period of at least 8 hours.

### Exemplary Methods of Use

In one embodiment, the present invention relates to a method of treating a condition of a subject in need thereof, comprising the steps of

applying to an affected area of the subject an effective amount of a foam prepared from any one of the above-mentioned compositions.

In one embodiment, the present invention relates to any one of the above-mentioned methods, further comprising the step of:

expelling from an aerosol container any one of the above-mentioned compositions, thereby preparing a foam.

In one embodiment, the present invention relates to a method of treating a condition of a subject in need thereof, comprising the steps of:

applying to an affected area of the subject an effective amount of a foam prepared from any one of the above-mentioned compositions, thereby simultaneously treating and moisturizing the affected area.

In one embodiment, the present invention relates to a method of treating a condition of a subject in need thereof, comprising the steps of:

applying to an affected area of the subject an effective amount of a foam prepared from any one of the above-mentioned compositions, thereby simultaneously treating and hydrating the affected area.

In one embodiment, the present invention relates to the above-mentioned method, wherein the condition is acne, decubital ulcers, stasis ulcers, burns, varicose ulcers, sycosis vulgaris, or seborrhea.

In one embodiment, the present invention relates to any one of the above-mentioned methods, wherein the condition is acne.

In one embodiment, the present invention relates to any one of the above-mentioned methods, wherein the subject is human.

In one embodiment, the present invention relates to the above-mentioned method, wherein the affected area of the subject is the face, neck, décolletage, or back. In one embodiment, the present invention relates to the above-mentioned method, wherein the affected area of the subject is the face. In one embodiment, the present invention relates to the above-mentioned method, wherein the affected area of the subject is the décolletage or back.

In one embodiment, the present invention relates to any one of the above-mentioned methods, wherein the composition is applied once daily.

In one embodiment, the present invention relates to any one of the above-mentioned methods, wherein the composition is applied twice daily.

### Exemplary Methods of Formulation

In one embodiment, the invention relates to a method, comprising the steps of: heating water in a first container;

adding glycerol, propylene glycol, methylparaben, propyl paraben, and disodium EDTA to the first container, thereby making an aqueous mixture;

heating the aqueous mixture until the aqueous mixture reaches about 70° C. to about 75° C., thereby forming an aqueous solution;

mixing cetostearyl alcohol, emulsifying wax, steareth-10, dimethicone, C₁₂-C₁₅ alkyl benzoates, and butylated hydroxytoluene in a second container, thereby forming a nonaqueous mixture;

heating the nonaqueous mixture until the nonaqueous mixture reaches about 70° C. to about 75° C., thereby forming a nonaqueous solution;

adding the aqueous solution to the nonaqueous solution, thereby forming a first mixture;

homogenizing and cooling the first mixture until the first mixture reaches from about 40° C. to about 45° C., thereby forming a second mixture;

mixing and cooling the second mixture until the second mixture reaches about 30° C., thereby forming a third mixture;

adding benzoyl peroxide to the third mixture and stirring the third mixture, thereby forming a fourth mixture; and

optionally homogenizing the fourth mixture.

In one embodiment, the invention relates to a method, comprising the steps of:

heating water in a first container;

adding glycerol, propylene glycol, methylparaben, propyl paraben, citric acid, sodium citrate, and disodium EDTA to the first container, thereby making an aqueous mixture;

heating the aqueous mixture until the aqueous mixture reaches about 70° C. to about 75° C., thereby forming an aqueous solution;

mixing cetostearyl alcohol, emulsifying wax, steareth-10, dimethicone, C₁₂-C₁₅ alkyl benzoates, and butylated hydroxytoluene in a second container, thereby forming a nonaqueous mixture;

heating the nonaqueous mixture until the nonaqueous mixture reaches about 70° C. to about 75° C., thereby forming a nonaqueous solution;

adding the aqueous solution to the nonaqueous solution, thereby forming a first mixture;

homogenizing and cooling the first mixture until the first mixture reaches from about 40° C. to about 45° C., thereby forming a second mixture;

mixing and cooling the second mixture until the second mixture reaches about 30° C., thereby forming a third mixture;

adding benzoyl peroxide to the third mixture and stirring the third mixture, thereby forming a fourth mixture; and

optionally homogenizing the fourth mixture.

In one embodiment, the present invention relates to the above-mentioned method, further comprising the step of

placing the fourth mixture in an aerosol container.

In one embodiment, the present invention relates to the above-mentioned method, further comprising the step of:

purging the aerosol container with an inert gas, wherein the inert gas is argon or nitrogen.

In one embodiment, the present invention relates to the above-mentioned method, further comprising the step of:

adding a propellant to the aerosol container, wherein the propellant is a hydrofluoroalkane.

### EXEMPLIFICATION

### Example 1

### Effects of Stability-Enhancing Measures

Multiple formulations (A-H) of benzoyl peroxide were made in order to study the effect of antioxidants or an argon purge on the stability of the formulations (Table 1). The antioxidant blend contained the following composition: butylated hydroxytoluene (BHT) (21.5 g, 86%), propyl gallate (2.5 g, 10%), and t-butylhydroquinone (TBHQ) (1.0 g, 4%). Formulations C, D, G, and H were purged with argon for 2 min.

Upon formulation, the samples were put into glass jars with aerosol valves and stored under various temperatures (4° C., 25° C., 30° C., and 40° C.).

TABLE 1
BPO Excipient Compatibility Experiments -
HFA 134a Formulations

| Formu- | BPO | Water | Antiox- | | HFA 134a |
|---|---|---|---|---|---|
| lation | (g) | (g) | idant (g) | Argon | (g) |
| A | 1 | 9 | | | |
| B | 1 | 9 | 0.1 | | |
| C | 1 | 9 | | Purge | |
| D | 1 | 9 | 0.1 | Purge | |
| E | 1 | 9 | | | 12 |
| F | 1 | 9 | 0.1 | | 12 |
| G | 1 | 9 | | Purge | 12 |
| H | 1 | 9 | 0.1 | Purge | 12 |

Incorporation of inert atmosphere, specifically argon gas, in the aerosol can headspace improved BPO stability and slowed the rate of BPO degradation in the presence of 1,1,1,2-tetrafluoroethane (HFA 134a) propellant. Excipient compatibility experiments demonstrated a 7.6% increase in BPO stability after storage under accelerated conditions (3 months at 40° C. and 75% relative humidity (RH)) (compare formulation H to formulation E). Table 1 shows the design of the excipient compatibility study and Table 2 shows the results, demonstrating the improvement of BPO stability.

TABLE 2
Benzoyl Peroxide Excipient Compatibility
Experiments - HFA 134a Formulations
(% Label Claim - 3 months at 40° C.)

| Formulation | 1 month % LC | 2 month % LC | 3 month % LC |
|---|---|---|---|
| A | 97.9 | 98.5 | 97.7 |
| B | 95.8 | 92.7 | 84.5 |
| C | 98.7 | 98.5 | 97.8 |
| D | 92.6 | 88.1 | 82.9 |
| E | 89.8 | 80.3 | 69.2 |
| F | 88.1 | 80.9 | 73.9 |
| G | 84.4 | 82.1 | 59.1 |
| H | 89.7 | 84.1 | 76.8 |

### Example 2

### General Formulation Procedure

A 5% BPO foam concentrate was formulated. The quantities used are depicted in Table 3. A similar procedure may be used to formulate any other BPO formulation.

TABLE 3
5% Benzoyl Peroxide Foam Concentrate Formulation.

| | | Calculated | Actual quantity |
|---|---|---|---|
| W | % w/w | quantity(g) | (g) |
| Cetostearyl Alcohol USP | 2.50 | 25.0 | 25.04 |
| Emulsifying Wax USP | 2.50 | 25.0 | 25.08 |
| Steareth-10 | 1.00 | 10.0 | 10.09 |
| Dimethicone | 1.00 | 10.0 | 10.05 |
| BHT | 0.05 | 0.5 | 0.52 |
| C₁₂-C₁₅ Alkyl Benzoates | 0.50 | 5.0 | 5.02 |
| Purified Water, USP | 76.95 | 769.5 | 769.50 |
| Glycerol | 7.50 | 75.0 | 75.20 |
| Propylene Glycol USP | 2.50 | 25.0 | 25.19 |
| Methylparaben | 0.30 | 3.0 | 3.01 |
| Propylparaben | 0.10 | 1.0 | 1.02 |
| Disodium EDTA | 0.10 | 1.0 | 1.09 |
| Benzoyl Peroxide | 5.00 | 50.0 | 50.00 |

The concentrate was made as follows: purified water (705.5 g) was weighed in a beaker. The beaker was then heated on a hotplate. Glycerol (75.0 g), propylene glycol (2.50 g), methylparaben (3.0 g), propylparaben (1.0 g), and disodium ethylenediaminetetraacetic acid (disodium EDTA) (1.0 g) were added to the warm beaker. The mixture was heated to 70-75° C., until all solids were dissolved, thus forming the aqueous phase.

Separately, cetostearyl alcohol (25.0 g), emulsifying wax (25.0 g), steareth-10 (10.0 g), dimethicone (10.0 g), C₁₂-C₁₅ alkyl benzoates (5.0 g), and butylated hydroxytoluene (BHT) (0.5 g) were weighed in a beaker. The beaker was heated to 70-75° C. to dissolve, melt, and mix the contents uniformly, thus forming the oil phase.

The aqueous phase was added to the oil phase and the mixture homogenized for 5 minutes with a Silverson Homogenizer set at speed 6.5. The emulsion was then cooled with the outside cold water jacket to 40-45° C. while mixing at dial 5.5. The Silverson homogenizer was removed and a lightning mixer was used, with continued cooling. Once the mixture reached 30° C., cooling was halted and thoroughly-mixed benzoyl peroxide (50.0 g) (in the form of BPO 42 USP gel, 114 g) was added slowly with rapid stirring. The mixture was mixed until dispersed. If necessary, the Silverson homogenizer may be used to disperse the BPO for 5-10 minutes at speed 5.5.

### Example 3

### Effects of BPO Concentration, Propellant Identity, and Environmental Engineering Controls on Stability of Formulations

In addition, a series of aerosol foam formulations of benzoyl peroxide (5 and 10%) were prepared with three propellant systems. A stability study was set up to determine the effect of engineering controls (removing oxygen from product concentrate and an argon purge of can headspace) on the stability of benzoyl peroxide and the formulation physical stability. Tables 4 and 5 summarize the BPO aerosol foam formulations and Tables 6-9 summarize the three-month stability results at all temperatures (4, 25, 30, and 40° C.).

TABLE 4
BPO Concentrates
(Manufactured with Engineering Controls)

| | J | K | L | M | N | P |
|---|---|---|---|---|---|---|
| Cetostearyl Alcohol USP | 2.50 | 2.50 | 2.50 | 2.50 | 2.50 | 2.50 |
| Emulsifying Wax USP | 2.50 | 2.50 | 2.50 | 2.50 | 2.50 | 2.50 |
| Steareth-10 | 1.00 | 1.00 | 1.00 | 1.00 | 1.00 | 1.00 |
| Dimethicone | 1.00 | 1.00 | 1.00 | 1.00 | 1.00 | 1.00 |
| BHT | 0.05 | 0.05 | 0.05 | 0.05 | 0.05 | 0.05 |
| C12-C15 Alkyl Benzoates | 0.50 | 0.50 | 0.50 | 0.50 | 0.50 | 0.50 |
| Purified Water, USP | 76.95 | 76.95 | 76.95 | 71.95 | 71.95 | 71.95 |
| Glycerol | 7.50 | 7.50 | 7.50 | 7.50 | 7.50 | 7.50 |
| Propylene Glycol USP | 2.50 | 2.50 | 2.50 | 2.50 | 2.50 | 2.50 |
| Methylparaben | 0.30 | 0.30 | 0.30 | 0.30 | 0.30 | 0.30 |
| Propylparaben | 0.10 | 0.10 | 0.10 | 0.10 | 0.10 | 0.10 |
| Disodium EDTA | 0.10 | 0.10 | 0.10 | 0.10 | 0.10 | 0.10 |
| Benzoyl Peroxide | 5.00 | 5.00 | 5.00 | 10.00 | 10.00 | 10.00 |
| Target BPO (%) | 5.00 | 5.00 | 5.00 | 10.00 | 10.00 | 10.00 |
| Propellent - HFA | 134a | 227 | 134a/227 | 134a | 227 | 134a/227 |

TABLE 5
BPO Concentrates
(Manufactured without Engineering Controls)

| | Q | R | S | T | U | V |
|---|---|---|---|---|---|---|
| Cetostearyl Alcohol USP | 2.50 | 2.50 | 2.50 | 2.50 | 2.50 | 2.50 |
| Emulsifying Wax USP | 2.50 | 2.50 | 2.50 | 2.50 | 2.50 | 2.50 |
| Steareth-10 | 1.00 | 1.00 | 1.00 | 1.00 | 1.00 | 1.00 |
| Dimethicone | 1.00 | 1.00 | 1.00 | 1.00 | 1.00 | 1.00 |
| BHT | 0.05 | 0.05 | 0.05 | 0.05 | 0.05 | 0.05 |
| C12-C15 Alkyl Benzoates | 0.50 | 0.50 | 0.50 | 0.50 | 0.50 | 0.50 |
| Purified Water, USP | 76.95 | 76.95 | 76.95 | 71.95 | 71.95 | 71.95 |
| Glycerol | 7.50 | 7.50 | 7.50 | 7.50 | 7.50 | 7.50 |
| Propylene Glycol USP | 2.50 | 2.50 | 2.50 | 2.50 | 2.50 | 2.50 |
| Methylparaben | 0.30 | 0.30 | 0.30 | 0.30 | 0.30 | 0.30 |
| Propylparaben | 0.10 | 0.10 | 0.10 | 0.10 | 0.10 | 0.10 |
| Disodium EDTA | 0.10 | 0.10 | 0.10 | 0.10 | 0.10 | 0.10 |
| Benzoyl Peroxide | 5.00 | 5.00 | 5.00 | 10.00 | 10.00 | 10.00 |
| Target BPO (%) | 5.00 | 5.00 | 5.00 | 10.00 | 10.00 | 10.00 |
| Propellent - HFA | 134a | 227 | 134a/227 | 134a | 227 | 134a/227 |

Improved BPO stability was demonstrated for the aerosol foam formulations prepared with engineering controls (compare Table 6 with Table 7; and Table 8 with Table 9). It was surprisingly discovered that the fluorinated propellant 1,1,1,2,3,3,3-heptafluoropropane (HFA 227) improved the chemical stability of the BPO aerosol foam formulations when compared to the foams containing HFA 134a or an HFA 134a/227 propellant blend. In each case, the propellant was 11.76% by weight of the final composition in the aerosol can; the BPO concentrate, as formulated in Tables 4 and 5, makes up the remaining 88.24% by weight of the final composition.

Additionally, the physical stability of the benzoyl peroxide foam formulation was affected by the engineering controls. As an example, 10% BPO aerosol foam (formulation T, without engineering controls), when stored at 40° C. for 3 months, dispensed as a low viscosity liquid, indicating physical instability of the product. In contrast, 10% BPO aerosol foam (Formulation M, with engineering controls), when stored at 40° C. for 3 months, dispensed as a cosmetically-elegant foam, acceptable for commerce.

TABLE 6
Three-Month Stability Results for 5% BPO Aerosol Foam Formulations
(Manufactured with Engineering Controls)

| | | | | |
|---|---|---|---|---|
| Initial | 4° C. | 25° C. | 30° C. | 40° C. |

5% BPO Foam - 3M Stability

| | | | | | |
|---|---|---|---|---|---|
| J (134a) | 104.2 | 103.8 | 101.5 | 101.4 | 78.2 |
| K (227) | 99.6 | 99.8 | 98.5 | 97.5 | 88.3 |
| L (134a/227) | 102.0 | 103.3 | 100.5 | 100.7 | 81.7 |

5% BPO Foam - 3M Stability (% change)

| | | | | | |
|---|---|---|---|---|---|
| J | N/A | −0.4 | −2.7 | −2.8 | −26.0 |
| K | N/A | 0.2 | −1.1 | −2.1 | −11.3 |
| L | N/A | 1.3 | −1.5 | −1.3 | −20.3 |

TABLE 7
Three-Month Stability Results for 5% BPO Aerosol Foam Formulations
(Manufactured without Engineering Controls)

| | | | | |
|---|---|---|---|---|
| Initial | 4° C. | 25° C. | 30° C. | 40° C. |

5% BPO Foam - 3M Stability

| | | | | | |
|---|---|---|---|---|---|
| Q (134a) | 96.2 | 96.6 | 94.0 | 89.5 | 64.0 |
| R (227) | 98.9 | 100.1 | 97.2 | 96.8 | 87.5 |
| S (134a/227) | 98.9 | 95.9 | 96.8 | 97.0 | 75.3 |

5% BPO Foam - 3M Stability (% change)

| | | | | | |
|---|---|---|---|---|---|
| Q | N/A | 0.4 | −2.2 | −6.7 | −32.2 |
| R | N/A | 1.2 | −1.7 | −2.1 | −11.4 |
| S | N/A | −3.0 | −2.1 | −1.9 | −23.6 |

TABLE 8
Three-Month Stability Results for 10% BPO Aerosol Foam Formulations
(Manufactured with Engineering Controls)

| | | | | |
|---|---|---|---|---|
| Initial | 4° C. | 25° C. | 30° C. | 40° C. |

10% BPO Foam - 3M Stability

| | | | | | |
|---|---|---|---|---|---|
| M (134a) | 100.8 | 100.8 | 99.1 | 100.0 | 72.0 |
| N (227) | 100.4 | 97.4 | 97.9 | 96.7 | 92.0 |
| P (134a/227) | 96.4 | 96.1 | 96.1 | 94.0 | 74.9 |

10% BPO Foam - 3M Stability (% change)

| | | | | | |
|---|---|---|---|---|---|
| M | N/A | 0.0 | −1.7 | −0.8 | −28.8 |
| N | N/A | −3.0 | −2.5 | −3.7 | −8.4 |
| P | N/A | −0.3 | −0.3 | −2.4 | −21.5 |

TABLE 9
Three-Month Stability Results for 10% BPO Aerosol Foam Formulations
(Manufactured without Engineering Controls)

| | | | | |
|---|---|---|---|---|
| Initial | 4° C. | 25° C. | 30° C. | 40° C. |

10% BPO Foam - 3M Stability

| | | | | | |
|---|---|---|---|---|---|
| T (134a) | 96.3 | 88.9 | 98.4 | 95.4 | 57.7 |
| U (227) | 89.4 | 89.7 | 88.9 | 90.3 | 83.9 |
| V (134a/227) | 93.4 | 90.6 | 83.2 | 71.7 | 75.6 |

10% BPO Foam - 3M Stability (% change)

| | | | | | |
|---|---|---|---|---|---|
| T | N/A | −7.4 | 2.1 | −0.9 | −38.6 |
| U | N/A | 0.3 | −0.5 | 0.9 | −5.5 |
| V | N/A | −2.8 | −10.2 | −21.7 | −17.8 |

### Example 4

### Color Analysis

Incorporation of inert atmosphere, specifically argon gas, in the aerosol can headspace decreased color formation in the aqueous 10% BPO/HFA 134a aerosol system. Formulation E (from Table 1) developed a dark brown color after storage under accelerated conditions (30° C.). Formulation G (from Table 1), which contained a thorough purge of an inert gas, specifically argon, demonstrated a significant reduction in color formation after storage under accelerated conditions (30° C.), and was clear. FIGS. 1 and 2 demonstrate the effect of inert gas (argon) on the color of the aqueous 10% BPO/HFA 134a system.

### Example 5

### Effects of Buffer on BPO Concentration, Reduced Color Formation and Physical Stability of Formulations

A series of buffered aerosol foam formulations of benzoyl peroxide (5.3%) were prepared with one propellant system (134a). A stability study was set up to determine the effect of buffer concentration and type on the stability of benzoyl peroxide, reduced color formation and the physical stability of the formulations. Formulations were prepared similarly to the previous examples, as described in Table 4 (with engineering controls). Sodium Citrate/Citric Acid and Sodium Phosphate Monobasic/Sodium Hydroxide buffers were utilized to achieve a pH of 6. Table 10 summarizes the buffered aerosol foam formulations of benzoyl peroxide.

TABLE 10
Buffered Aerosol Foam Formulations of Benzoyl Peroxide
(Manufactured with Engineering Controls)

| | W | X | Y | Z |
|---|---|---|---|---|
| Cetostearyl Alcohol USP | 2.25 | 2.25 | 2.25 | 2.25 |
| Emulsifying Wax USP | 2.25 | 2.25 | 2.25 | 2.25 |
| Steareth-10 | 0.9 | 0.9 | 0.9 | 0.9 |
| Dimethicone | 0.9 | 0.9 | 0.9 | 0.9 |
| BHT | 0.05 | 0.05 | 0.05 | 0.05 |
| C12-C15 Alkyl Benzoates | 0.45 | 0.45 | 0.45 | 0.45 |
| Purified Water, USP | 76.73 | 76.41 | 76.78 | 76.47 |
| Glycerol | 7.50 | 7.50 | 7.50 | 7.50 |
| Propylene Glycol USP | 2.50 | 2.50 | 2.50 | 2.50 |
| Methylparaben | 0.30 | 0.30 | 0.30 | 0.30 |
| Propylparaben | 0.10 | 0.10 | 0.10 | 0.10 |
| Disodium EDTA | 0.10 | 0.10 | 0.10 | 0.10 |
| Sodium Citrate | 0.62 | 0.93 | 0.00 | 0.00 |
| Citric Acid | 0.05 | 0.06 | 0.00 | 0.00 |
| Sodium Phosphate Monobasic | 0.00 | 0.00 | 0.62 | 0.93 |
| Sodium Hydroxide (10% Solution) | 0.00 | 0.00 | q.s. to pH 6 | q.s. to pH 6 |
| Benzoyl Peroxide | 5.30 | 5.30 | 5.30 | 5.30 |
| Target BPO (%) | 5.30 | 5.30 | 5.30 | 5.30 |
| Propellant - HFA | 134a | 134a | 134a | 134a |
| Propellant - HFA (%) | 12.49 | 12.49 | 12.49 | 12.49 |

Formulations W-Z demonstrated a similar benzoyl peroxide stability profile as example J in Table 6. This demonstrates that the addition of pharmaceutically acceptable buffers does not impact benzoyl peroxide stability in the aerosol foam formulations. It was discovered that the addition of buffer further reduced the color formation of the formulations (on stability). It was also discovered that the addition of buffer helped to stabilize the viscosity of the formulations, resulting in the improved dispensing of the formulation from the aerosol container closure system. The previous un-buffered example J in Table 6 showed an increase in viscosity on stability and demonstrated difficulty in shaking and dispensing (clogging) from the aerosol container closure system.

### Example 6

### Effects of Various Concentrations of BPO Foam Compositions on Skin Redness

Subjects were treated with a blank, an aerosol foam vehicle, a 3.3% BPO aerosol foam, or a 5.3% BPO aerosol foam for five days. A Konica Minolta CR-400 Chroma Meter was used each day to measure the a*-value of the treated area of each subject. The a*-value used was a measure of the red-green scale in CIE L*a*b* (CIELAB) Color Space. Negative a*-values indicate green, while positive a*-values indicate red or magenta. The data demonstrate no significant difference in the a*-value between all treatments. The data also demonstrate no statistically-significant difference in a*-value over time for each treatment. Redness is an indication of skin irritation or erythema. See FIG. 3.

### Example 7

### Comparison of Various BPO Topical Formulations on Stratum Corneum Moisture Values

Subjects (n=10) were treated with 5.3% BPO aerosol foam, 5% BPO/1% Clindamycin gel, or 5% BPO gel, and the level of hydration of the stratum corneum in the treated area was measured over time. The data demonstrate the superior hydrating or moisturizing properties of 5.3% BPO aerosol foam versus commercially available treatments. The aerosol foam produces a statistically significant difference (p<0.05) in hydration at the 95% confidence interval. See FIG. 4.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition, comprising a concentrate and a propellant, wherein
the concentrate comprises
an amount of benzoyl peroxide, wherein the amount of benzoyl peroxide is about 0.5% to about 15% by weight of the concentrate;
an amount of water, wherein the amount of water is about 55% to about 95% by weight of the concentrate; and
an amount of an antioxidant or a preservative, wherein the amount of the antioxidant or the preservative is about 0.01% to about 5% by weight of the concentrate; and
the propellant is a hydrofluoroalkane propellant.

2. The composition of claim 1, wherein the hydrofluoroalkane propellant is 1,1,1,2-tetrafluoroethane, 1,1,1,2,3,3,3-heptafluoropropane, or a mixture thereof.

3. The composition of claim 1, wherein the antioxidant or the preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, sodium ascorbate, ascorbyl palmitate, propyl gallate, disodium EDTA, and mixtures thereof.

4. The composition of claim 1, wherein the antioxidant or the preservative is methylparaben, propylparaben, butylated hydroxytoluene, disodium EDTA, citric acid, sodium citrate, or a mixture thereof.

5. The composition of claim 1, wherein the concentrate further comprises an emollient or a moisturizer; and the emollient or the moisturizer is present in an amount from about 5% to about 20% by weight of the concentrate.

6. The composition of claim 5, wherein the emollient or the moisturizer is selected from the group consisting of glycerol, PPG-15 stearyl ether, lanolin alcohol, lanolin, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, lactic acid, butylene glycol, sodium PCA, carbowax 200, carbowax 400, carbowax 800, and mixtures thereof.

7. The composition of claim 5, wherein the emollient or the moisturizer is glycerol, dimethicone, C₁₂-C₁₅ alkyl benzoates, propylene glycol, or a mixture thereof.

8. The composition of claim 1, wherein the concentrate further comprises an emulsifier or a surfactant; and the emulsifier or the surfactant is present in an amount from about 2% to about 10% by weight of the concentrate.

9. The composition of claim 8, wherein the emulsifier or the surfactant is selected from the group consisting of polysorbate 20, polysorbate 40, polysorbate 60, polysorbate 80, steareth-10, sodium dodecyl sulfate, lauryl dimethyl amine oxide, cetyltrimethylammonium bromide (CTAB), polyoxyethylene sorbitan, octoxynol, N,N-dimethyldodecylamine-N-oxide, hexadecyltrimethylammonium bromide (HTAB), polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, behentrimonium methosulfate, and mixtures thereof.

10. The composition of claim 8, wherein the emulsifier or the surfactant is cetostearyl alcohol, emulsifying wax, steareth-10, or a mixture thereof.

11. The composition of claim 1, wherein the composition is colorless.

12. The composition of claim 1, wherein the composition is in an aerosol container, thereby forming a headspace of the aerosol container; and the headspace of the aerosol container is substantially free of oxygen.

13. The composition of claim 1, wherein the composition is in an aerosol container, thereby forming a headspace of the aerosol container; and the headspace of the aerosol container consists essentially of argon.

14. The composition of claim 1, wherein the composition is in an aerosol container; and the composition is expelled as a foam when the aerosol container is actuated.

15. The composition of claim 14, wherein the foam is non-irritating when applied to the skin of a subject.

16. The composition of claim 14, wherein the foam is moisturizing for a period of at least 8 hours when applied to the skin of a subject.

17. A composition, consisting essentially of a concentrate and a propellant, wherein
the concentrate consists essentially of
from about 0.5% to about 15% benzoyl peroxide by weight of the concentrate;
from about 55% to about 95% water by weight of the concentrate; and
from about 0.01% to about 5% of a first mixture by weight of the concentrate, wherein the first mixture comprises butylated hydroxytoluene, methylparaben, propylparaben, citric acid, sodium citrate, or disodium EDTA;
from about 5% to about 20% of a second mixture by weight of the concentrate, wherein the second mixture comprises dimethicone, C₁₂-C₁₅ alkyl benzoates, glycerol, or propylene glycol; and
from about 2% to about 10% of a third mixture by weight of the concentrate, wherein the third mixture comprises cetostearyl alcohol, emulsifying wax, or steareth-10; and
the propellant is a hydrofluoroalkane propellant, wherein the hydrofluoroalkane propellant is 1,1,1,2-tetrafluoroethane or 1,1,1,2,3,3,3-heptafluoropropane.

18. A composition, consisting essentially of a concentrate and a propellant, wherein
the concentrate consists essentially of
about 5.3% benzoyl peroxide by weight of the concentrate;
about 76.73% water by weight of the concentrate;
about 0.05% butylated hydroxytoluene by weight of the concentrate;
about 0.3% methylparaben by weight of the concentrate;
about 0.1% propylparaben by weight of the concentrate;
about 0.1% disodium EDTA by weight of the concentrate;
about 0.62% sodium citrate by weight of the concentrate;
about 0.05% citric acid by weight of the concentrate;
about 0.9% dimethicone by weight of the concentrate;
about 0.45% C₁₂-C₁₅ alkyl benzoates by weight of the concentrate;
about 7.5% glycerol by weight of the concentrate;
about 2.5% propylene glycol by weight of the concentrate;
about 2.25% cetostearyl alcohol by weight of the concentrate;
about 2.25% emulsifying wax by weight of the concentrate; and
about 0.9% steareth-10 by weight of the concentrate; and
the propellant is 1,1,1,2-tetrafluoroethane.

19. A method, comprising the steps of
heating water in a first container;
adding glycerol, propylene glycol, methylparaben, propyl paraben, citric acid, sodium citrate, and disodium EDTA to the first container, thereby making an aqueous mixture;
heating the aqueous mixture until the aqueous mixture reaches about 70° C. to about 75° C., thereby forming an aqueous solution;
mixing cetostearyl alcohol, emulsifying wax, steareth-10, dimethicone, C₁₂-C₁₅ alkyl benzoates, and butylated hydroxytoluene in a second container, thereby forming a nonaqueous mixture;
heating the nonaqueous mixture until the nonaqueous mixture reaches about 70° C. to about 75° C., thereby forming a nonaqueous solution;
adding the aqueous solution to the nonaqueous solution, thereby forming a first mixture;
homogenizing and cooling the first mixture until the first mixture reaches from about 40° C. to about 45° C., thereby forming a second mixture;
mixing and cooling the second mixture until the second mixture reaches about 30° C., thereby forming a third mixture;
adding benzoyl peroxide to the third mixture and stirring the third mixture, thereby forming a fourth mixture;
optionally homogenizing the fourth mixture; and
placing the fourth mixture in an aerosol container.

20. The method of claim 19, further comprising the step of
purging the aerosol container with an inert gas, wherein the inert gas is argon or nitrogen.

21. The method of claim 19, further comprising the step of
adding a propellant to the aerosol container, wherein the propellant is a hydrofluoroalkane.

22. A method of treating a condition in a subject in need thereof, comprising the steps of
applying to an affected area of the subject an effective amount of a foam prepared from a composition of claim 1.

23. The method of claim 22, further comprising the step of:
expelling from an aerosol container a composition of claim 1, thereby preparing a foam.

24. The method of claim 22, wherein the condition is acne, decubital ulcers, stasis ulcers, burns, varicose ulcers, sycosis vulgaris, or seborrhea.

25. The method of claim 22, wherein the subject is a human.

26. The method of claim 25, wherein the affected area of the subject is the face, neck, décolletage, or back.

27. The method of claim 25, wherein the composition is applied once daily or twice daily.
